(19) United States
(12) Patent Application Publication
(10) Pub. No.: US 2021/0386428 A1
(43) Pub. Date: Dec. 16, 2021

(54) ERGONOMIC SURGICAL INSTRUMENT FOR MINIMALLY INVASIVE SURGERY

(71) Applicant: FlexLogical ApS, København K (DK)

(72) Inventors: Frederik Emil Cederfeldt LARSEN, Copenhagen K (DK); Peter SØLBECK, Vedbæk (DK)

(73) Assignee: FlexLogical ApS, København K (DK)

(21) Appl. No.: 17/283,194

(22) PCT Filed: Oct. 2, 2019
(86) PCT No.: PCT/EP2019/076675

§ 371 Date: Apr. 6, 2021

(30) Foreign Application Priority Data
Oct. 12, 2018 (DK) PA201800717

(51) Int. Cl. A61B 17/128; A61B 17/122

(52) U.S. Cl. CPC A61B 17/1285; A61B 2017/00424; A61B 17/122

(57) ABSTRACT

A surgical instrument including a handle, a hollow shaft, and an end effector coupled to a distal end of the hollow shaft, the end effector being operatively connected to the handle through operating means extending within the hollow shaft. A multiaxial joint couples a distal end of the handle to a proximal end of the hollow shaft, the multiaxial joint allowing multiaxial rotation of the hollow shaft in relation to the handle. The multiaxial rotation includes rotation of the hollow shaft around a center axis of the hollow shaft, and rotation in at least two planes in relation to the distal of the handle.

### TECHNICAL FIELD

The aspects of the disclosed embodiments relate to a surgical instrument for minimally invasive surgery, said surgical instrument comprising a handle, an end effector which is operatively connected to the handle, and a hollow shaft connecting the handle to the end effector.

### BACKGROUND

Minimally invasive surgery requires surgical instruments which engage tissue through small incisions or natural orifices in the body of a patient, and often reduces the recovery time and amount of complications in comparison with traditional open surgery.

Minimally invasive surgery also requires some versatility to be inbuilt in the instrument, such that the user has the best possible conditions for engaging tissue. These conditions include allowing the user to be able to hold the handle of the surgical instrument ergonomically, e.g. allowing the user to maintain his/her wrist straight.

EP1836986 discloses a surgical instrument comprising a ball-and-socket joint connecting the handle to the shaft, allowing the handle to be somewhat pivoted in two planes of motion relative the shaft. The end effector is connected to the handle by means of four wires extending equidistantly along the outside of the shaft. As the handle is pivoted, one opposite pair of wires interact to cause the end effector to mimic and move simultaneously with the handle. Hence, the end effector mimics the movement of the handle, which mimicking movement includes corresponding movement, whereby the end effector moves in the same direction and orientation as the handle, and mirrored movement, whereby the end effector moves in an opposite direction and orientation as the handle. The control over the end effector is improved, however the ergonomic situation for the user is not significantly improved. Hence, there is a need for providing an improved surgical instrument which allows the user to operate the instrument without bending or otherwise straining the wrist.

### SUMMARY

It is an object to provide an improved surgical instrument. The foregoing and other objects are achieved by the features of the independent claims. Further implementation forms are apparent from the dependent claims, the description, and the figures.

According to a first aspect, there is provided a surgical instrument comprising a handle, a hollow shaft, an end effector coupled to a distal end of the hollow shaft, the end effector being operatively connected to the handle through operating means extending within the hollow shaft, a multiaxial joint coupling a distal end of the handle to a proximal end of the hollow shaft, the multiaxial joint allowing multiaxial rotation of the hollow shaft in relation to the handle, the multiaxial rotation including rotation of the hollow shaft around a center axis of the hollow shaft, and rotation in at least two planes in relation to the distal end of the handle.

This surgical instrument allows the user to be able to place and hold the handle of the surgical instrument ergonomically, e.g. allowing the user to maintain his/her wrist straight, relative the shaft and end effector, which is comfortable for an extended period of time. Such a solution also allows the user to adjust the location of the handle around multiple axes, thereby allowing the user to place the handle in any comfortable position while maintaining the shaft and the end effector in a correct working position. Furthermore, the instrument becomes more flexible which in turn improves the reach of the instrument and, subsequently, the user.

In a possible implementation form of the first aspect, the handle is rotatable around the center axis, and the handle is rotatable in two planes in relation to the proximal end of the hollow shaft, the two planes extending parallel with or perpendicular to the center axis and perpendicular to each other, allowing the handle to be placed in at least any desired two-dimensional position relative the shaft, within certain limitations.

In a further possible implementation form of the first aspect, the hollow shaft is rotatable around the center axis and in three perpendicular planes simultaneously, allowing the handle to be placed in any desired three-dimensional position relative the shaft, within certain limitations.

In a further possible implementation form of the first aspect, the multiaxial rotation does not affect the position of the end effector in relation to the position of the handle, which allows the secure and stable placement of the end effector to be maintained regardless of possible movement of the handle.

In a further possible implementation form of the first aspect, the operating means comprises at least one of a wire, a hose, and a rod, the operating means extending through an interior of the multiaxial joint and through the distal end of the handle, into an interior of the handle, such that the operating means is completely protected from the exterior.

In a further possible implementation form of the first aspect, the handle comprises at least one grip part and one actuating part, the actuating part being pivotally connected to the grip part and operatively connected to the end effector.

In a further possible implementation form of the first aspect, the operating means comprises an axially extending rod and a first pivot link, the first pivot link being arranged within the interior of the handle and being operatively connected to the rod, the grip part, and the actuating part, facilitating operation of the end effector simultaneously with operation of the handle.

In a further possible implementation form of the first aspect, a proximal end of the rod is connected to a proximal end of the first pivot link, the proximal end of the first pivot link further being slidingly connected to the grip part, and a distal end of the first pivot link being pivotally connected to the actuating part, allowing the end effector to be operated without applying much stress onto the handle.

In a further possible implementation form of the first aspect, the multiaxial joint is a spherical joint comprising first and second at least partially spherical shells, open ends of the first and second shells having mating surfaces such that there is a common overlap area, comprising the open ends, between the first shell and the second shell when an open end of one of the first shell and the second shell is pressed into the open end of the other of the first shell and the second shell, allowing the handle to be rotated in relation to the shaft over a significant part of the surface of one of the spherical shells.

In a further possible implementation form of the first aspect, one of the first and second shells is integral with at least one of the grip part(s) or connected to at least one of the grip part(s).

In a further possible implementation form of the first aspect, the common overlap area changes size as the first and second shells are rotated in relation to each other, in at least one of the planes, allowing an as large rotation as possible between the two shells.

In a further possible implementation form of the first aspect, at least one of the first and second shells is provided with a hollow guiding tube, protruding from at least one of an outer surface and an inner surface of the first and second shells, and accommodating at least one of the proximal end of the hollow shaft, the operating means, and a connection to the handle, the hollow guiding tube providing additional support and protection for the components accommodated within the hollow guiding tube as well as assisting in aligning e.g. the operating means with connection to the handle.

In a further possible implementation form of the first aspect, the surgical instrument further comprises a bearing arranged between the hollow guiding tube and the hollow shaft, allowing the hollow shaft to rotate around the center axis without affecting the position of the handle.

In a further possible implementation form of the first aspect, at least one mating surface of the first and second shells is provided with at least one of a microstructure, a coating, and a roughening for increased friction in the common overlap area, further increasing the level of interlocking between the first and second shells.

In a further possible implementation form of the first aspect, the mating surface (of the first shell is provided with a first microstructure and the mating surface of the second shell is provided with a second microstructure, the first microstructure and the second microstructure comprising one of a plurality of recesses and at least one protrusion, the protrusion(s) being adapted for engaging and interlocking with the recesses, facilitating a secure lock between the first and second shells preventing the shells from rotating relative each other.

In a further possible implementation form of the first aspect, one of the first microstructure (and the second microstructure is provided on a radially displaceable tongue, allowing a simple and releasable interlock between the shells.

In a further possible implementation form of the first aspect, at least one of the first and second shells is provided with at least one slit extending from the open end of the first and/or second shells, allowing tongues to be formed in between the slits which tongues can flex radially outwards, allowing one shell to be pushed over the surface of the other shell.

In a further possible implementation form of the first aspect, the multiaxial joint further comprises a third shell arranged around a periphery of the first and second mating shells, in the common overlap area, a locking lever protruding from the third shell, the third shell being moveable between an unlocked position and a locked position by operating the locking lever, allowing the first and second shells to be interlocked.

In a further possible implementation form of the first aspect, in the unlocked position, diameters of the first and second shells are unaffected by the third shell allowing the first and second shells to be rotated in relation to each other, and wherein, in the locked position, the diameters of at least one of the first and second shells are reduced in at least the common overlap area such that the first and second shells cannot be rotated in relation to each other. This allows the handle to be not only rotated, but also locked in a rotated position, in relation to the shaft. Thus, this implementation allows the user to lock the handle in a comfortable position.

In a further possible implementation form of the first aspect, an inner surface of said third shell or an outer surface of a radially outermost positioned shell of the first and second shells comprises a peripherally extending, radially directed groove, the radial depth of the groove decreasing in a direction towards the open end of the first, second, or third shell such that the groove comprises a maximum bottom thickness section and a minimum bottom thickness section, the third shell being in the unlocked position when one of the radially outermost positioned shells and the third shell is at least partially arranged within the minimum bottom thickness section, and the third shell being in the locked position when one of the radially outermost positioned shells and the third shell is at least partially arranged within the maximum bottom thickness section. Hence, a locking function which is comprises as few interacting parts as possible is provided.

In a further possible implementation form of the first aspect, the radially displaceable tongue is formed in the radially outermost positioned shell of the first and second shells, wherein, in the unlocked position, a radial position of the radially displaceable tongue is unaffected by the third shell, and the first and second shells can be rotated in relation to each other, and wherein, in the locked position, the radially displaceable tongue is moved towards a radially innermost positioned shell of the first and second shells, the first microstructure and the second microstructure engaging in at least the common overlap area such that the first and second shells cannot be rotated in relation to each other.

In a further possible implementation form of the first aspect, one of the first and second shells is rotatably connected to the hollow shaft, and the other of the first and second shells is rigidly connected to the handle, and wherein, when the third shell is in the locked position, the handle is locked to the multiaxial joint, facilitating secure and intentional multiaxial rotation.

In a further possible implementation form of the first aspect, the surgical instrument further comprises a joint housing, a proximal end of the joint housing being connected to the handle and a distal end of the joint housing being connected to the first or the second shell, the joint housing covering the multiaxial joint and the proximal end of the hollow shaft, providing additional support and protection for the components accommodated within the joint housing.

In a further possible implementation form of the first aspect, the joint housing shares a center axis with the hollow shaft, and the joint housing is partially rotatable around the center axis, providing a degree of flexibility to the joint housing such that the handle can be rotated essentially uninhibitedly.

In a further possible implementation form of the first aspect, the joint housing comprises a flexible part extending between at least first and second rigid parts, in the direction of the center axis, the first rigid part being connected to the first or second shell, the second rigid part being connected to the handle, facilitating secure connections between components while still allowing the handle to be rotated.

In a further possible implementation form of the first aspect, at least one of the first and second rigid parts comprises a plurality of grooves extending in the direction of the center axis, each groove comprising an end surface extending at an angle to the center axis, the end surface being arranged at a proximal end of the first rigid part and/or at a distal end of the second rigid part, the flexible part filling the grooves such that a flexible part surface corresponding to the end surface is formed in each groove. This allows for a tight and secure interface between the flexible part and the rigid parts.

In a further possible implementation form of the first aspect, the end surface comprises an opening through which the flexible part extends, the opening extending generally parallel with the center axis, providing a simple yet rigid connection between the flexible part and the rigid parts such that the flexible part cannot easily be separated from the rigid parts.

In a further possible implementation form of the first aspect, a section of the flexible part extends on a radially outer side and/or a radially inner side of the first rigid part and a section of the flexible part extends on a radially inner side and/or a radially outer side of the second rigid part, facilitating a more stable joint housing.

In a further possible implementation form of the first aspect, at least one of the first and second rigid parts have a truncated cone shape, the first rigid part having an outer diameter and/or an inner diameter which is smaller than the outer diameter and/or an inner diameter of the second rigid part, facilitating a joint housing having a shape which essentially corresponds to the shape of the multiaxial joint as far as allowing as small outer dimensions as possible.

In a further possible implementation form of the first aspect, a proximal end of the second rigid part comprises locking means adapted for interacting with at least one corresponding recess in the handle, such that a secure and simple interconnection, which does not limit the rotation, is provided between the handle and the joint.

In a further possible implementation form of the first aspect, the locking means is one of a peripheral flange and a snap lock.

In a further possible implementation form of the first aspect, the handle and the operating means are configured to actuate the end effector between an open position and a closed position, a proximal end of the end effector being coupled to a distal end of the hollow shaft, the end effector comprising a first leg and a second leg, the second leg being pivotally connected to the first leg at a distal end of the end effector, the second leg extending at an acute angle to the first leg when the end effector is in the open position, and the second leg extending generally parallel with the first leg when the end effector is in the closed position. Such a solution facilitates an end effector which is safe to insert through an incision or an opening into a body, since the legs of the end effector, should they be in an open configuration, are automatically closed to a closed configuration such that they cannot get accidentally caught in tissue. Furthermore, the surgical instrument is actuated only by means of the handle, dispensing with the need for external instruments.

In a further possible implementation form of the first aspect, the second leg comprises cutting means and the first leg comprises a recess for accommodating the cutting means when the end effector is in the closed position, facilitating a safe cutting application having a significantly reduced risk of accidental cutting of tissue.

In a further possible implementation form of the first aspect, the second leg comprises a staple magazine, facilitating a further application for the surgical instrument.

In a further possible implementation form of the first aspect, the distal end of the end effector comprises protection lips extending between the first leg and the second leg, the protection lips preventing tissue from getting stuck in the connection between the first leg and the second leg.

In a further possible implementation form of the first aspect, the surgical instrument further comprises at least one support rod, a proximal end of the support rod being pivotally connected to the proximal end of the end effector, the support rod extending at an acute angle to the first leg when the end effector is in the open position, the support rod extending in parallel with the first leg when the end effector is in the closed position. The support rod assures that tissue clamped between the legs of the end effector cannot accidentally slide out from between the legs.

In a further possible implementation form of the first aspect, the operating means comprises an axially extending rod and a second pivot link, a distal end of the rod being coupled to a distal end of the second leg by means of the second pivot link, a first end of the second pivot link being pivotally connected to the axially extending rod and a second end of the second pivot link being pivotally connected to the second leg. This facilitates a reliable way of operating the end effector which does not require any additional space.

In a further possible implementation form of the first aspect, the second leg is pivotally suspended from the rod at a first position on the second leg, and the second end of the pivot link is pivotally connected to the second leg at a second position spaced from the first position, allowing the second leg to be maneuvered.

In a further possible implementation form of the first aspect, the second pivot link comprises a plate extending between the first end and the second end, the first end being connected to the rod by a first hinge pin, the second end being connected to the second leg by a second hinge pin, and the second leg being hinged to the hollow shaft by a third hinge pin.

In a further possible implementation form of the first aspect, the second leg has a free proximal end and a hinged distal end, and the first position is at or adjacent the hinged distal end. This solution facilitates an end effector which is safe to insert through an incision or an opening into a body, while also allowing an as large range of reach as possible for the user of the instrument.

In a further possible implementation form of the first aspect, the second position is nearer to the hinged distal end than to the free proximal end.

In a further possible implementation form of the first aspect, the third hinge pin is arranged in or adjacent the center axis of the hollow shaft.

In a further possible implementation form of the first aspect, the second pivot link is operatively connected to the rod at or near the proximal end or the distal end of the rod.

In a further possible implementation form of the first aspect, the second leg is configured to rotate towards the hollow shaft when the rod is translated in a direction away from the third hinge pin and the second leg is configured to rotate away from the hollow shaft when the rod is translated in a direction towards the third hinge pin, allowing the second leg to close completely or almost completely against the first leg.

In a further possible implementation form of the first aspect, the rod is partially enclosed by the first leg of the end effector, the end effector is actuated from the open position to the closed position when the rod is moved in a direction away from the distal end of the end effector, the end effector being actuated from the closed position to the open position when the rod is moved in a direction towards the distal end of the end effector. This facilitates reliable operating means which comprise as few parts as possible while still providing sufficient force to actuate the end effector between positions also when tissue is arranged within the end effector.

In a further possible implementation form of the first aspect, the operating means further comprises a piston and a spring, the rod being partially enclosed by the piston and the spring, the spring applying a force onto a proximal end of the piston in a direction towards the distal end of the end effector, the rod and the piston being axially moveable in relation to at least one of each other and the end effector, facilitating operating means which can not only actuate the end effector but also move e.g. ligation clips to a correct position.

In a further possible implementation form of the first aspect, the rod and the piston are releasably connected such that the rod and the piston are axially moveable as one unit, in relation to the end effector, allowing the operating means to be multifunctional yet still reliable.

In a further possible implementation form of the first aspect, the rod and the piston are releasably connected by means of a piston lock, the piston lock comprising a plurality of locking grooves arranged in the rod, and a corresponding locking protrusion arranged at a distal end of the piston, allowing the surgical instrument to be operated, allowing the components of the operating means to be interlocked by means of a simple and reliable solution which is easy to implement.

In a further possible implementation form of the first aspect, the end effector is adapted for at least one of photographing and manipulating tissue, facilitating a surgical instrument having a wide range of applications.

According to a second aspect, there is provided a surgical system comprising the above described surgical instrument and at least one ligation clip, the end effector of the surgical instrument being configured to enclose the ligation clip such that a distal end of the ligation clip is arranged adjacent the distal end of the end effector and a proximal end of the ligation clip is arranged adjacent the proximal end of the end effector, the ligation clip comprising a first ligation clip leg and a second ligation clip leg, the first ligation clip leg and the second ligation clip leg being pivotally connected at the distal end of the ligation clip, the first ligation clip leg and the second ligation clip leg comprising locking means for interlocking the first and second ligation clip legs at the proximal end of the ligation clip in a position where the first ligation clip leg and the second ligation clip leg are superimposed onto each other, wherein, when the end effector is in an initial open position, the ligation clip has an open configuration where the second ligation clip leg extends at an acute angle to the first ligation clip leg, when the end effector has been actuated to a closed position, the ligation clip has a closed configuration where the first and second ligation clip legs are superimposed onto each other, when the end effector is returned to an open position, the ligation clip remains in the closed configuration by means of the locking means.

Such a system facilitates a surgical instrument which is safe to insert through an incision or an opening into a body, since the legs of the end effector, should they be in an open configuration, are automatically closed to a closed configuration such that they cannot get accidentally caught in tissue. Furthermore, the system is actuated only by means of the handle, dispensing with the need for external instruments.

In a possible implementation form of the second aspect, comprising a plurality of ligation clips, in open configuration and arranged successively between the distal end of the end effector and the handle, all ligation clips but a first ligation clip located adjacent the distal end of the end effector being enclosed by the first leg of the end effector and the rod, allowing the surgical instrument to be charged with several ligation clips and, hence, the surgical system to be used to ligate several locations in one session without having to recharge between separate ligation instances.

In a further possible implementation form of the second aspect, the ligation clips are arranged in abutment such that axial displacement of any ligation clip enclosed by the first leg and the rod induces a corresponding displacement of all ligation clips located between the ligation clip and the distal end of the end effector, providing a simple way of moving a sequence of ligation clips from a storage position to a position of use.

According to a third aspect, there is provided a method of operating the surgical system, wherein the operating means comprises at least an axially extending rod and wherein a first ligation clip is arranged within the end effector such that the first ligation clip is enclosed by the first leg of the end effector and the rod, the first ligation clip having an open configuration, the method comprising the steps of displacing the first ligation clip in a direction towards the distal end of the end effector, to a position where the first ligation clip is enclosed by the first and second legs of the end effector and the end effector is in an open position, by moving the rod in a direction towards the distal end of the end effector, closing the first ligation clip by actuating the end effector to a closed position by moving the rod in a direction away from the distal end of the end effector, and moving the rod in a direction towards the distal end of the end effector, such that the end effector is returned to the open position, allowing the first ligation clip, having a closed configuration, to be released from the end effector.

By means of this method, there is provided an end effector which is safe to insert through an incision or an opening into a body, since the legs of the end effector, should they be in an open configuration, are automatically closed to a closed configuration such that they cannot get accidentally caught in tissue. Furthermore, the system is actuated only by means of the handle, dispensing with the need for external instruments for e.g. closing the end effector or removing a closed ligation clip.

In a possible implementation form of the third aspect, the operating means further comprises a piston and a spring, a piston lock comprising rod locking grooves and a piston locking protrusion interlocking the rod and the piston in a first locking position when the end effector is returned to the open position, a second ligation clip being arranged within the end effector such that the second ligation clip is enclosed by the first leg of the end effector and the rod, the second ligation clip having an open configuration the method further comprising the steps of moving the rod in a direction away from the distal end of the end effector, whereby the piston lock is released from the first locking position and the piston is allowed to move in relation to the rod, simultaneously actuating the end effector to the closed position, and the piston moving towards the end effector, by means of the spring applying force onto a proximal end of the piston until the piston lock engages a second locking position, a distal end of the piston being arranged closer to the distal end of the end effector when the piston lock is in the second locking position than in the first locking position, moving the rod in a direction towards the distal end of the end effector, the piston being moved along with the rod in the same direction, simultaneously actuating the end effector to the open position, moving the rod in a direction away from the distal end of the end effector, whereby the piston lock is released from the second locking position, the locking protrusion engaging a longitudinal groove arranged in the rod such that the piston is allowed to move in relation to the rod, simultaneously actuating the end effector to the closed position, the distal end of the piston engaging with the second ligation clip, pushing the second ligation clip in a direction towards the distal end of the end effector by means of the spring, until the second ligation clip, or a third ligation clip located between the second ligation clip and the distal end of the end effector, is enclosed by the first and second legs of the end effector, moving the rod in a direction towards the distal end of the end effector, simultaneously actuating the end effector to the open position. The method allows reliable operation of the surgical instrument, due to the provision of as few parts as possible which components still provide sufficient force to actuate the end effector between positions also when tissue is arranged within the end effector.

In a further possible implementation form of the third aspect, the method further comprises the steps of closing the second or third ligation clip, enclosed by the first and second legs of the end effector, by actuating the end effector to the closed position by moving the rod in a direction away from the distal end of the end effector, moving the rod in a direction towards the distal end of the end effector, such that the end effector is returned to the open position, allowing the second or third ligation clip, in the closed configuration, to be released from the end effector.

The method allows actuation only by means of the handle of the surgical instrument, dispensing with the need for external instruments for e.g. closing the end effector or removing a closed ligation clip.

This and other aspects will be apparent from and the embodiment(s) described below.

### BRIEF DESCRIPTION OF THE DRAWINGS

In the following detailed portion of the present disclosure, the aspects, embodiments, and implementations will be explained in more detail with reference to the example embodiments shown in the drawings, in which:

FIGS. 1a and 1b show a side view and a rear view of a surgical instrument in accordance with one embodiment of the present disclosure;

FIGS. 2a and 2b show a cross-sectional side view and a perspective exploded view of components of the surgical instrument in accordance with one embodiment of the present disclosure;

FIG. 2c shows a perspective exploded view of components of the surgical instrument in accordance with a further embodiment of the present disclosure;

FIGS. 3 shows a perspective exploded view of a multiaxial joint in accordance with one embodiment of the present disclosure;

FIGS. 4a and 4b show a side view and a perspective exploded view of a multiaxial joint, joint housing, and handle in accordance with an embodiment of the present disclosure;

FIG. 5a shows a perspective partial view of a handle in accordance with an embodiment of the present disclosure;

FIG. 5b shows a perspective partial view of a handle in accordance with a further embodiment of the present disclosure;

FIGS. 6a and 6b show a side view and a cross-sectional side view of an assembled multiaxial joint and joint housing in accordance with an embodiment of the present disclosure, the multiaxial joint being in an unlocked position;

FIGS. 7a and 7b show a side view and a cross-sectional side view of the assembled multiaxial joint and joint housing shown in FIGS. 5a and 5b, the multiaxial joint being in a locked position;

FIG. 8a shows a cross-sectional side view of components of the surgical instrument in accordance with an embodiment of the present disclosure;

FIG. 8b shows a cross-sectional side view of an assembled multiaxial joint and joint housing in accordance with an embodiment of the present disclosure;

FIGS. 9a to 9c show a perspective exploded view and a cross-sectional side view of components of the surgical instrument in accordance with a further embodiment of the present disclosure;

FIGS. 10a and 10b show a perspective view and a cross-sectional side view of a multiaxial joint in accordance with an embodiment of the present disclosure;

FIG. 10c shows a side view of the multiaxial joint shown in FIGS. 10a and 10b;

FIGS. 11a and 11b show perspective views of a multiaxial joint in accordance with an embodiment of the present disclosure;

FIGS. 12a and 12b show cross-sectional side views of the embodiment of

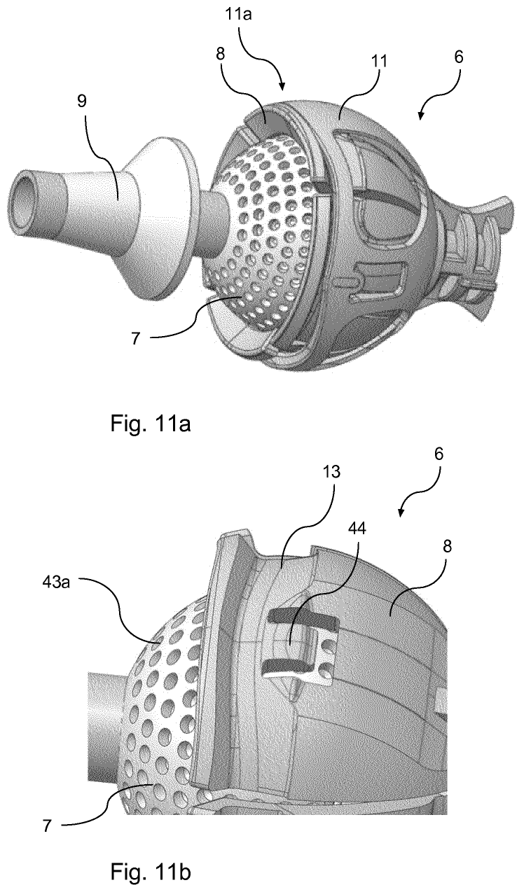

FIGS. 11a and 11b.

FIGS. 13a and 13b show a perspective view and a cross-sectional side view of a joint housing in accordance with an embodiment of the present disclosure;

FIG. 14a shows a side view of components of the joint housing shown in FIGS. 13a and 13b.

FIGS. 14b and 14c show perspective views of the components shown in FIG. 14a.

FIGS. 15a and 15b show detailed views of the interlocking between a flexible part and the components shown in FIGS. 14a-14c.

FIGS. 16a and 16b show a side view and a cross-sectional side view of a shaft, operating means, and an end effector in accordance with an embodiment of the present disclosure, the end effector being in an open position;

FIGS. 16c and 16d show a side view and a cross-sectional side view of the shaft, operating means, and end effector shown in FIGS. 16a and 16b, the end effector being in a closed position;

FIGS. 17a and 17b show perspective views of an end effector in accordance with an embodiment of the present disclosure, the end effector being in open position and closed position, respectively;

FIGS. 18a and 18b show perspective views of an end effector in accordance with a further embodiment of the present disclosure, the end effector being in open position and closed position, respectively;

FIG. 19 shows a side view of a ligation clip in accordance with an embodiment of the present disclosure;

FIGS. 20a and 20b show a perspective view and a cross-sectional side view of a shaft, operating means, and an end effector in accordance with a further embodiment of the present disclosure;

FIG. 20c shows a perspective exploded view of the shaft, operating means, and end effector shown in FIGS. 20a and 20b.

FIGS. 21a to 21h show partial side views of a surgical system and the function of the surgical system in accordance with an embodiment of the present disclosure;

FIGS. 22a and 22b show a side view and a cross-sectional side view of a shaft, operating means, and an end effector in accordance with a further embodiment of the present disclosure, the end effector being in an open position.

FIGS. 22c and 22d show a side view and a partial side view of the end effector shown in FIGS. 22a and 22b.

### DETAILED DESCRIPTION

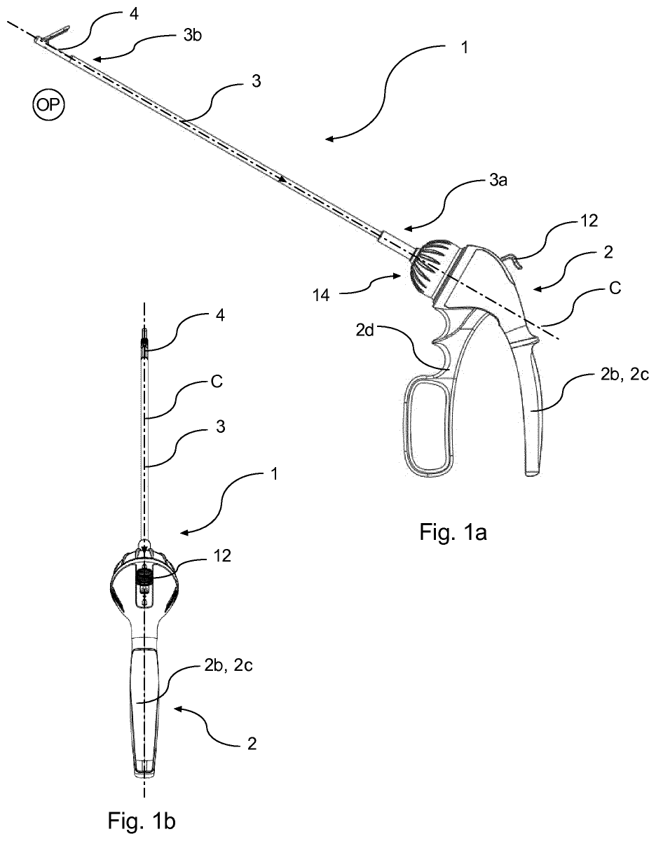

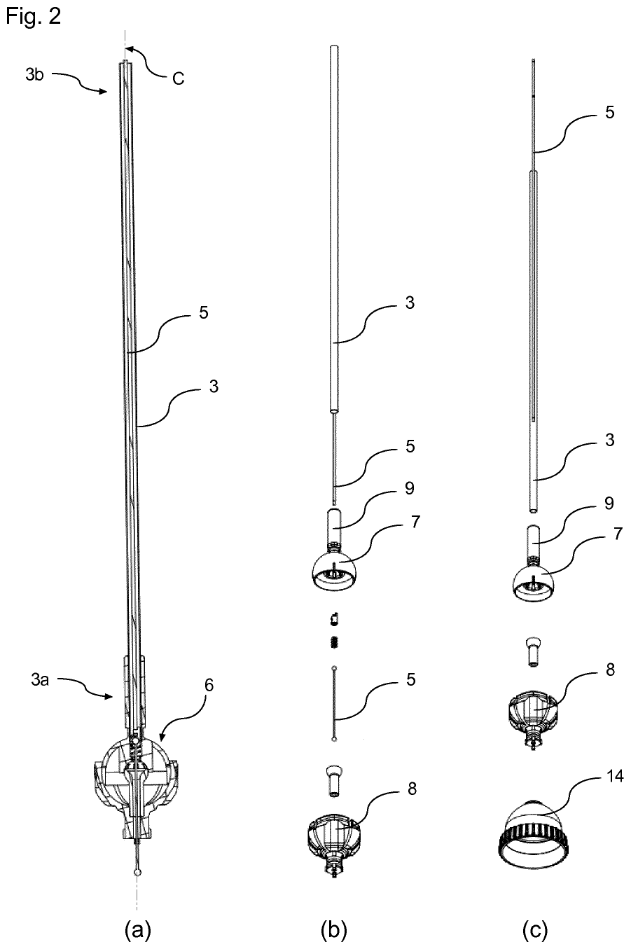

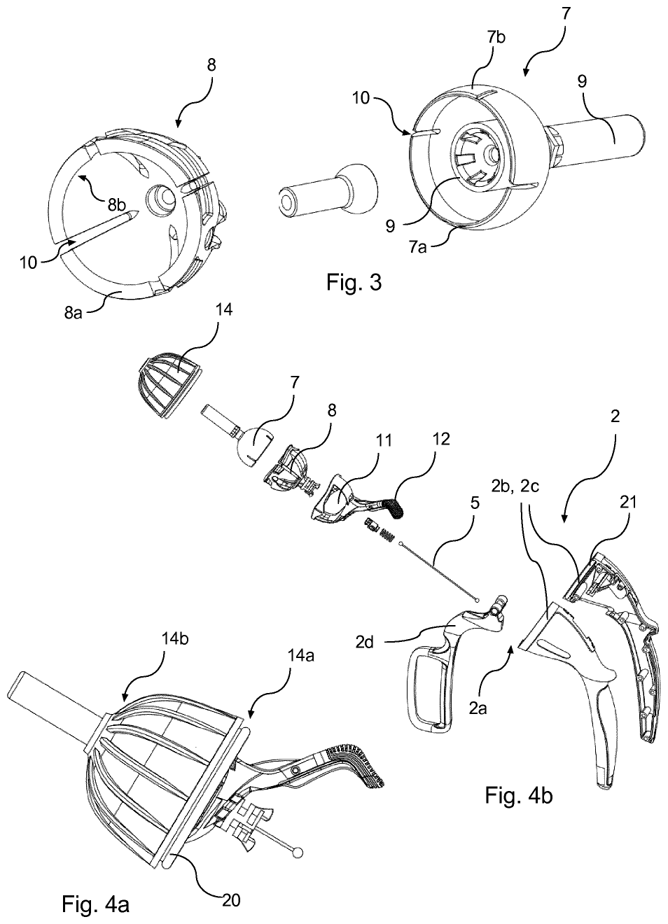

FIGS. 1a and 1b show a surgical instrument 1 comprising a handle 2, a hollow shaft 3, and an end effector 4. The hollow shaft 3 is an elongated body of a suitable material, such as e.g. stainless steel or carbon fiber reinforced composite material. A multiaxial joint 6, shown in more detail in FIGS. 3 to 12, couples a distal end 2a of the handle 2 to the proximal end 3a of the hollow shaft 3.

By “surgical instrument” is meant a device by means of which tissue of a patient is engaged by a further individual such as a surgeon. Throughout this application, the term “surgical instrument” includes devices such as prostheses or implants.

“Proximal” refers to the end of a component which is closest to the handle, and hence the individual holding the handle of the surgical instrument. Correspondingly, “distal” refers to the end of a component which is farthest away from the handle and the individual holding the handle.

As shown in FIGS. 1a-1b and 16a-16d, a proximal end 4a of the end effector 4 is coupled to a distal end 3b of the hollow shaft 3. The end effector 4 is adapted for at least one of photographing and manipulating tissue. “Manipulation” includes procedures such as severing, ligation clipping, injecting, burning, cutting, grasping, tearing, and stitching. One or several procedures may be executed simultaneously. “Tissue” includes veins, arteries, intestines, or umbilical cords (non-exhaustive lists).

The end effector 4 is operatively connected to the handle 2 through operating means 5 which partially extend within the hollow shaft 3, from the end effector 4 towards and past the proximal end 3a of the hollow shaft 3, through an interior of the multiaxial joint 6, and through the distal end 2a of the handle 2 and into the interior of the handle 2. The operating means 5 comprises at least one of a wire, a hose, and a rod. In one embodiment, the operating means 5 comprises a combination of a wire and a rod.

The handle 2 may be a squeeze handle or any other type of suitable handle, operated manually or by means of a robot. As shown in FIGS. 1a, 4b, and 5a-5b, the handle 2 may comprise at least one grip part 2b, 2c and one actuating part 2d, the actuating part 2d being pivotally connected to the grip part 2b, 2c and operatively connected to the end effector 4.

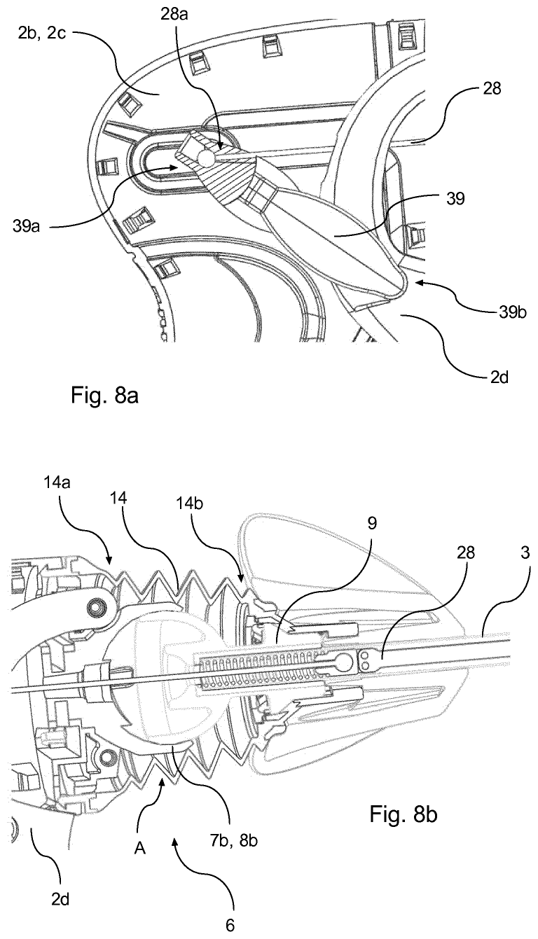

The operating means 5 may comprise an axially extending rod 28 and a first pivot link 39, as shown in FIG. 8a. The first pivot link 39 is arranged within the interior of the handle 2 and is operatively connected to the rod 28, the grip part 2b, 2c, and the actuating part 2d.

A proximal end 28a of the rod 28 is connected to a proximal end 39a of the first pivot link 39. The proximal end 39a of the first pivot link 39 is further slidingly connected to the grip part 2b, 2c. A distal end 39b of the first pivot link 39 is pivotally connected to the actuating part 2d.

As the actuating part 2d of the handle is pivoted towards the grip part 2b, 2c, the first pivot link 39 is also pivoted and the proximal end 39a of the first pivot link 39 moved in a direction towards a proximal end of the handle 2, i.e. in a direction away from the hollow shaft 3 and the end effector 4. Rod 28 is moved simultaneously, in the same direction, since proximal end 28a of the rod 28 is connected to the proximal end 39a of the first pivot link 39.

A multi-axial joint is a joint in which movement occurs around a plurality of axes and/or in a plurality of planes. The multiaxial joint 6 allows rotational movement of the hollow shaft 3 and end effector 4, in relation to the handle 2, around the center axis C of the hollow shaft 3, i.e. the entire hollow shaft 3 may be rotated around its own center axis. The multiaxial joint 6 also allows rotation in at least two planes around a distal end 2a of the handle. In one embodiment, at least one plane is not parallel with the center axis C the hollow shaft 3. The plane may extend at any angle to the center axis, such that the plane e.g. is perpendicular to the center axis. The multi-axial joint 6 may also allow rotational movement in planes that are parallel with the hollow shaft 3.

The multiaxial joint 6 allows multiaxial rotation of the hollow shaft 3 and end effector 4 in relation to the handle 2, which is substantially identical to allowing multiaxial rotation of the handle 2 in relation to the hollow shaft 3 and end effector 4. As previously mentioned, the multiaxial rotation includes rotation around the center axis C of the hollow shaft 3, the end effector 4 being rotated along with the hollow shaft 3. Furthermore, the multiaxial rotation does not affect the position of the end effector 4 in relation to the position of the handle 2, such that the end effector 4 remains in the same position when the handle 2 is pivoted relative the hollow shaft 3. In other words, the end effector 4 remains stationary and neither rotates nor pivots as the handle 2 is moved. Movement generated by the handle 2 and relating to the end effector 4 is limited to actuation of the end effector 4 between an open position OP and a closed position CP by means of actuating part 2d, which will be described in more detail farther below.

The hollow shaft 3 is rotatable around its own center axis C. In one embodiment the hollow shaft 3 is also rotatable in three planes simultaneously, in relation to the distal end 2a of the handle 2, such that free rotation is allowed in the three planes at the same time while preventing translation in any direction. In one embodiment, each plane extends parallel with or perpendicular to the center axis and perpendicular to each other. The hollow shaft 3 is rotatable around the center axis C and in the three planes simultaneously.

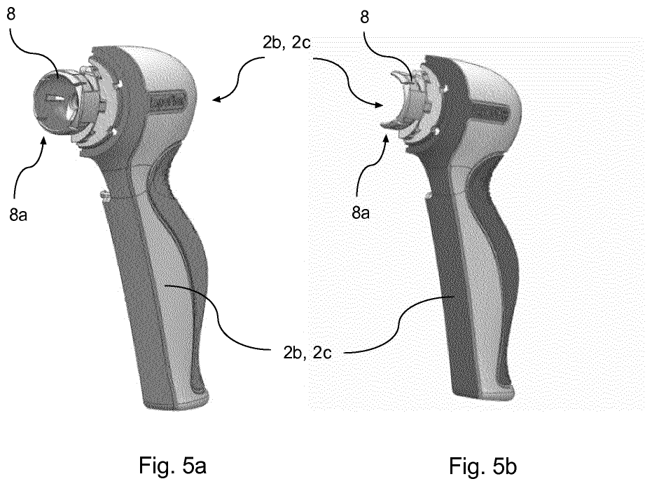

In one embodiment, the multiaxial joint 6 is a spherical joint comprising first 7 and second 8 partially spherical shells, each spherical shell comprising a spherical-shaped portion. In one embodiment, the first 7 or second 8 shell comprises a spherical segment formed by a pair of parallel planes cutting a hypothetical sphere with the center of the hypothetical sphere being arranged between the parallel planes. In a further embodiment, one of the parallel planes coincides with the center of the hypothetical sphere. In another further embodiment, both parallel planes extend on the same side of the center of the hypothetical sphere, i.e. one of the parallel planes is arranged between the center of the hypothetical sphere and the other parallel plane. Each shell 7, 8 may comprise of one integral part, as shown in FIG. 5a, or may comprise of several interconnected shell parts, as indicated in FIG. 5b which shows approximately one half of the second shell 8.

One of the first 7 and second 8 shells may be integral with at least one of the grip part(s) 2b, 2c. One of the first 7 and second 8 shells may also be connected to at least one of the grip part(s) 2b, 2c. FIG. 5a shows an embodiment wherein one of the second 8 shell is connected to one of the grip parts 2b, 2c.

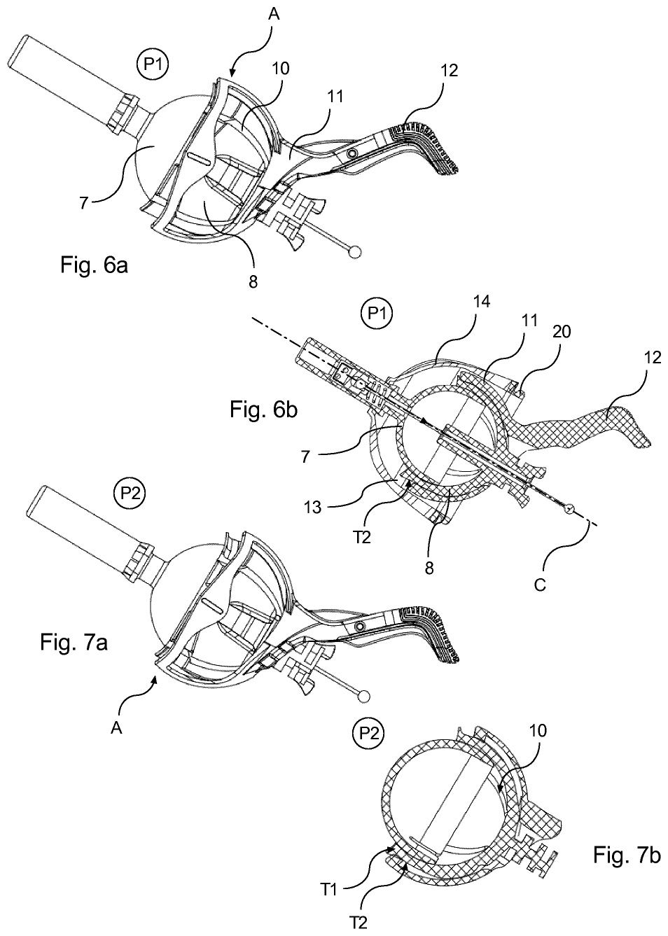

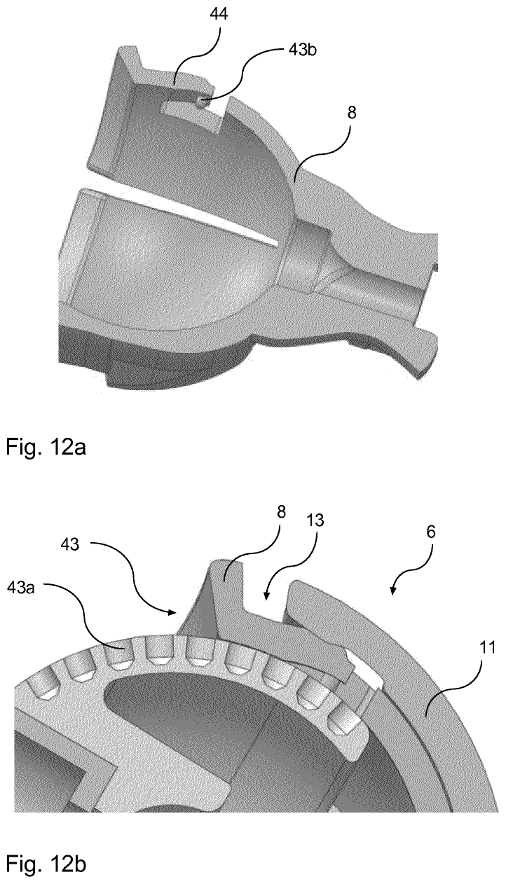

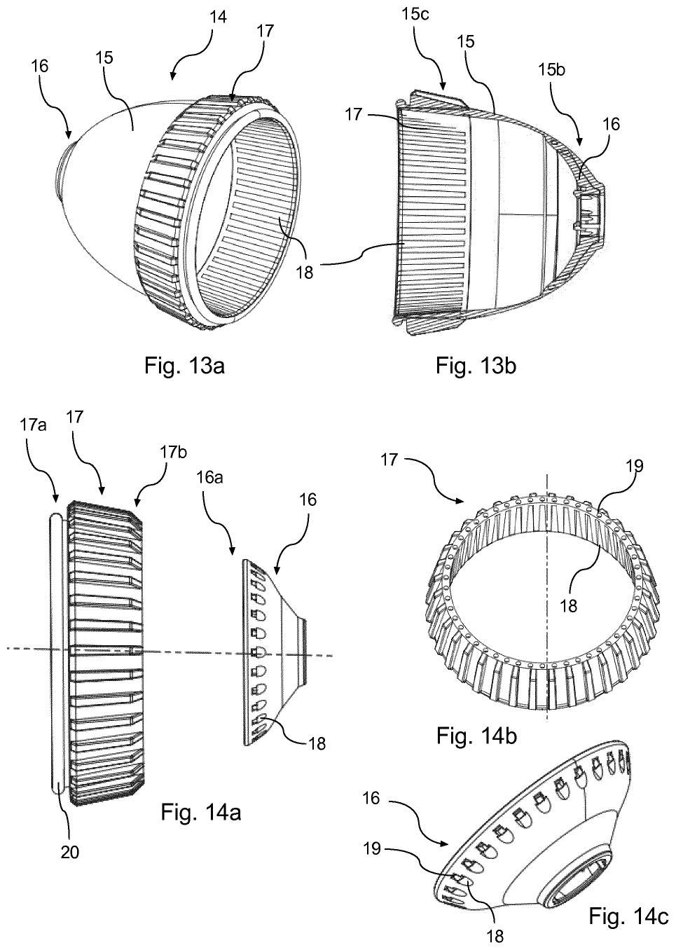

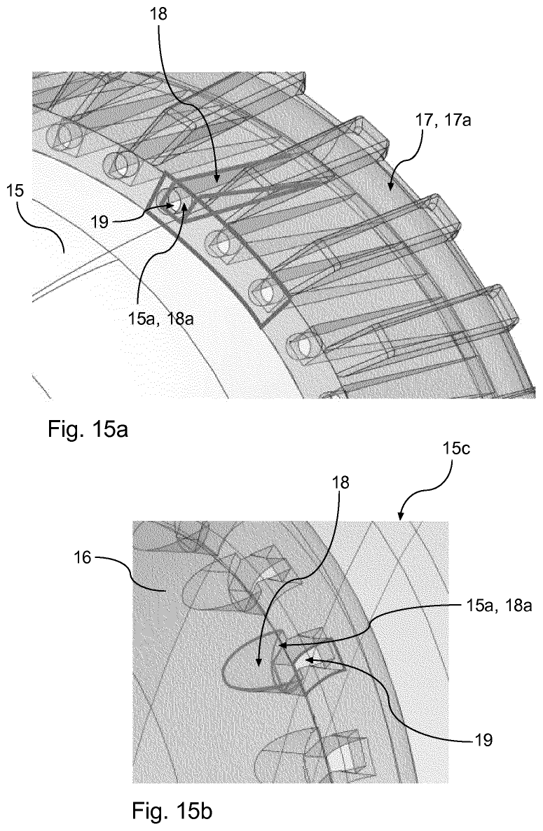

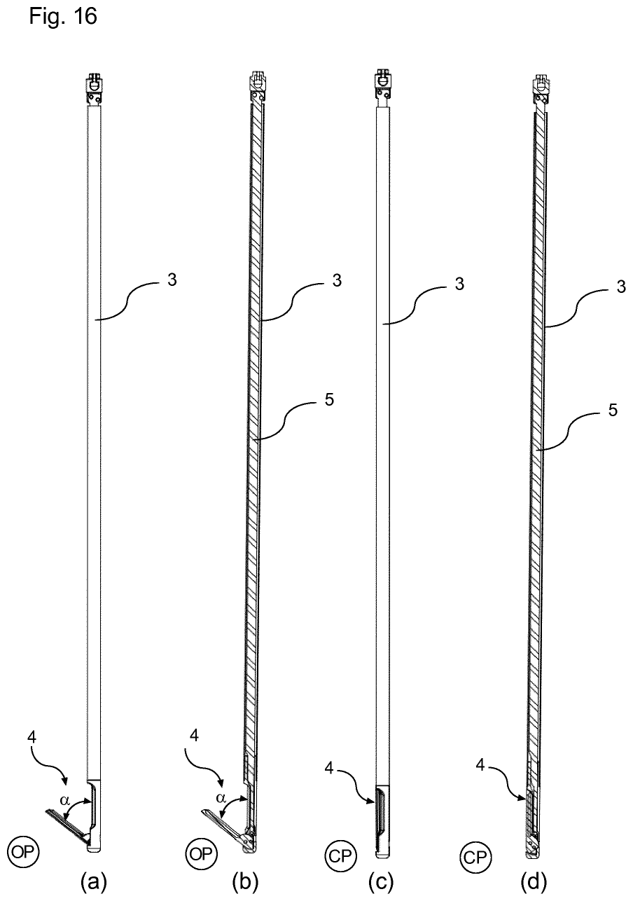

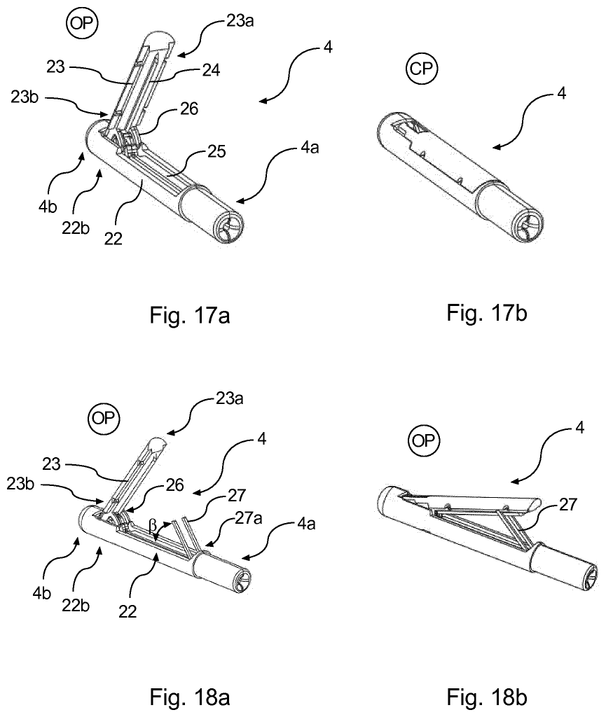

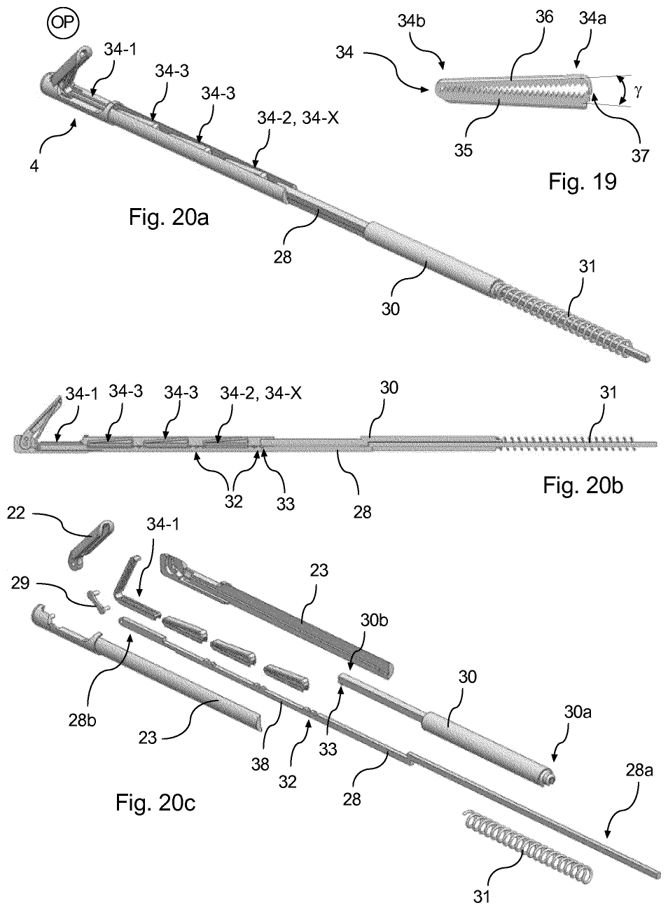

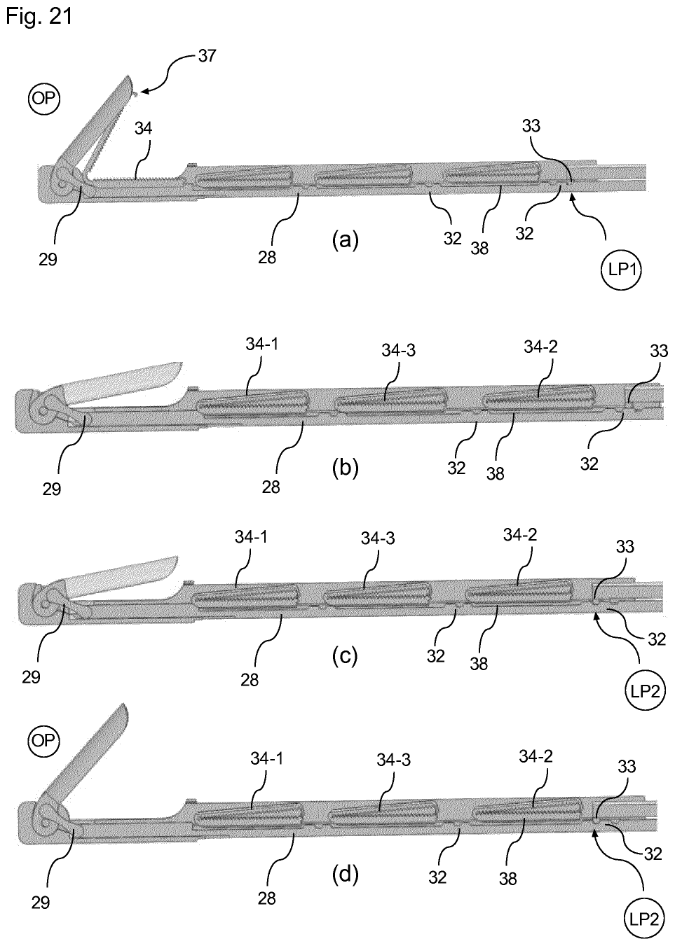

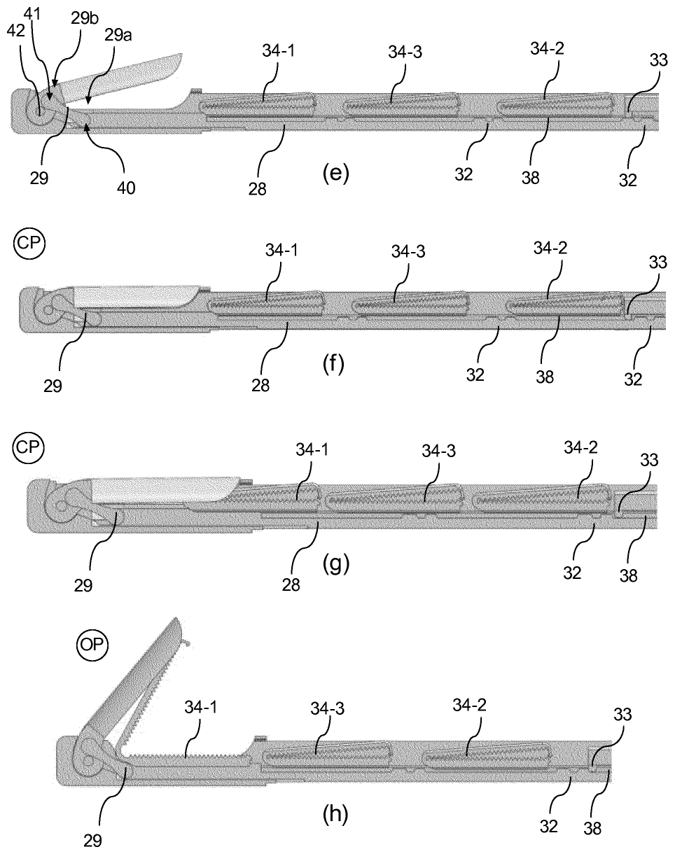

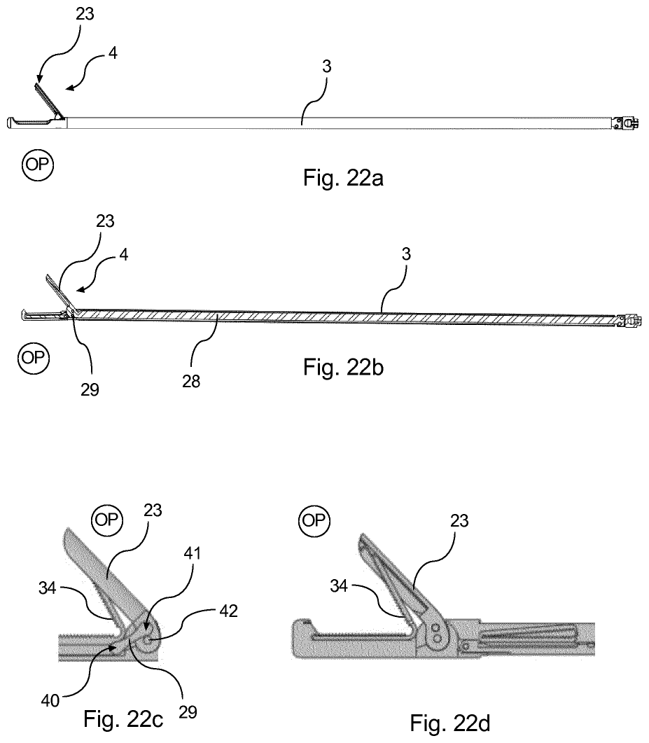

As shown in FIG. 3, the first shell 7 comprises an open end 7a, and the second shell 8 comprises an open end 8a. The first 7 and second 8 shells are arranged such that either the open end 7a of the first shell 7 overlaps the open end 8a of the second shell 8, or the open end 8a of the second shell 8 overlaps the open end 7a of the first shell 7. The overlap is shown in more detail in FIGS. 6a-7b, 8b, 10a-10c, 11a-11b, and 12b. The open end of one shell is pressed into the open end of the other shell, providing a so-called press fit between the two shells 7, 8, in a way which may allow the one shell to rotate in relation to the other shell, as shown in FIGS. 6a-7b and 10a-10c, or in a way which interlocks the two shells 7, 8 such that the one shell cannot rotate in relation to the other shell, as shown in FIGS. 11a-11b and 12b. In other words, the open ends 7a and 8a have mating surfaces 7b, 8b such that, when the first shell 7 and the second shell 8 mate, there is a common overlap area A between the first shell 7 and the second shell 8.

With regards solely to the movement of the first 7 and second 8 shells in relation to each other, in an embodiment in which the two shells 7,8, can be rotated in relation to each other, the multiaxial joint 6 may correspond to a ball-and-socket joint, wherein a ball moves within a socket so as to allow rotary motion in every direction within certain limits.

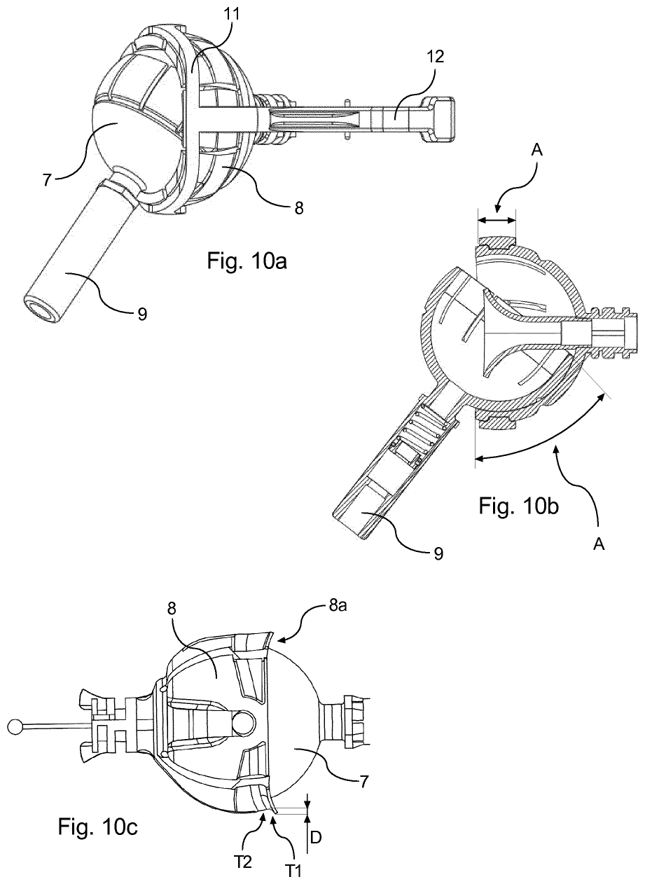

As the first 7 and second 8 shells are rotated in relation to each other, in at least one of the planes, the common overlap area A may change its size. Furthermore, the overlap area A at least partially changes its location. The first 7 and second 8 shells may both share a center axis C with the hollow shaft 3, i.e. as long as the handle 2 is not rotated in any of the three above-mentioned planes in relation to the proximal end 3a of the hollow shaft 3, and in which case the common overlap area A extends all around the periphery of both first 7 and second 8 shells. This position is shown in FIGS. 1a as well as 4a to 7a. When the first 7 and second 8 shells are rotated only around the center axis C, i.e. the shells 7, 8 remaining in the position shown in FIG. 6b, neither the size nor the location of the overlap area A changes. However, when the first 7 and second 8 shells are rotated in relation to each other in any of the three above-mentioned planes, as shown in FIGS. 10a and 10b, the common overlap area A extends only along a part of the periphery of both first 7 and second 8 shells.

One of the first 7 and second 8 shells is rotatably connected to the hollow shaft 3 and the other of the first 7 and second 8 shells is rigidly connected to the handle 2. For the sake of ease of reading, the text below will describe an embodiment wherein the first shell 7 is rotatably connected to the hollow shaft 3 and wherein the second shell 8 is rigidly connected to the handle 2. Furthermore, the first shell 7 will be described as the inner shell and the second shell 8 will be described as the outer shell, of the two partially overlapping shells. However, embodiments comprising an outer first shell and an inner second shell, as well as a first shell 7 rigidly connected to the handle 2 and a second shell 8 rotatably connected to the hollow shaft 3 are equally possible.

At least one of the first 7 and second 8 shells may be provided with a hollow guiding tube 9. The hollow guiding tube 9 protrudes from one or both of the outer surface and the inner surface of the first shell 7 and/or the second shell 8 shell. The hollow guiding tube 9 accommodates at least one of the proximal end 3a of the hollow shaft 3, a section of the operating means 5, and a connection to the handle 2.

As shown in FIG. 8b, a bearing 45 may be arranged between the hollow guiding tube 9 and the hollow shaft 3, allowing the hollow shaft 3 to rotate around the center axis C.

At least one of the first 7 and second 8 shells may be provided with throughgoing slits 10 extending from the open end 7a, 8a of the shell, which allows the shells 7,8 to overlap. By providing the second shell 8 with slits, tongues formed in between the slits can flex radially outwards, allowing the second shell 8 to be pushed over the surface of the first shell 7. Furthermore, the tongues can return to a radially more inward position as the second shell 8 is pushed farther across the first shell 7, in a direction from the open end 7a of the first shell, since the cross-sectional outer diameter of the first shell 7 decreases in the direction. This allows the first 7 and second 8 shells to interlock, at least to some extent, by means of press fit.

At least one of the mating surfaces 7b, 8b of the first 7 and second 8 shells, which surfaces together constitute the common overlap area A, may be provided with a microstructure 43, a coating and/or a roughening for increased friction in the common overlap area A, further increasing the level of interlocking between the first 7 and second 8 shells.

The microstructure 43 may comprise of interconnecting protrusions and recesses as shown in FIGS. 11a-11b and 12b.

In one embodiment, the mating surface 7b of the first shell 7 is provided with a first microstructure 43a, and the mating surface 8b of the second shell 8 is provided with a second microstructure (43b). The first microstructure 43a and the second microstructure 43b comprising one of a plurality of recesses and at least one protrusion, the protrusion(s) being adapted for engaging and interlocking with the recesses. FIGS. 11a-12b show embodiments wherein the first shell 7 is provided with a first microstructure 43a comprising a plurality of recesses, and the second shell 8 is provided with a second microstructure 43b comprising a protrusion.

One of the first microstructure 43a and the second microstructure 43b may be provided on a radially displaceable tongue 44. FIGS. 11b-12b show embodiments wherein the radially displaceable tongue 44, in an equilibrium state where it is not subjected to external pressure, protrudes slightly radially outwards, in a direction away from the innermost of the two shells 7, 8. This allows the two shells to rotate relative each other. As the tongue 44 is displaced radially inwards, towards the innermost shell, the first microstructure 43a and the second microstructure 43b eventually interconnect, preventing such rotation.

In one embodiment, the multiaxial joint 6 further comprises a third shell 11 arranged around the periphery of the first 7 and second 8 mating shells, in the common overlap area A. The third shell 11 is preferably essentially ring-shaped but may also have any other suitable configuration, such as e.g. a partially spherical or elliptical shell. The third shell 11 may have an inner diameter which is somewhat smaller than the largest outer diameter of the second shell 8, such that the third shell 11 can press the tongues on the second shell 8 inwards, toward the first shell 7, and subsequently interlock the first 7 and second 8 shells by means of press fit or mechanical locking means provided through the microstructure 43.

A locking lever 12 protrudes from the third shell 11, allowing the third shell 11 to be moveable between an unlocked position P1 and a locked position P2 as it is operated either manually or by means of a robot. The third shell is, during such operation, pivoted around the first 7 and second 8 shells such that one peripheral section of the third shell 11 is moved in a direction towards the open end 8a of the second shell 8 and the opposite peripheral section is moved from the open end 8a of the second shell 8. Depending on the actual position of the third shell 11, the first 7 and second 8 shells can be either interlocked or not interlocked in relation to each other. By interlocking the first 7 and second 8 shells, the position of the handle 2 is locked in relation to the shell which is connected to the proximal end 3a of the hollow shaft 3, preferably the second shell 8, allowing the person operating the surgical instrument to lock the instrument at any suitable angle.

The diameters of the first inner shell 7 and the outer second shell 8 remain unaffected by radial pressure from the third shell 11, such that the first 7 and second 8 shells can be rotated in relation to each other, when the third shell 11 is in the unlocked position P1.

The diameter of at least one of the first inner shell 7 and the outer second shell 8, preferably the outer second shell 8, is reduced by means of radial pressure applied by the third shell 11, at least in the common overlap area A, such that the inner surface of the outer second shell 8 presses against the outer surface of the inner first shell 7, preventing the first 7 and second 8 shells from rotating in relation to each other, when the third shell 11 is in the locked position

P2.

An inner surface of the third shell 11 or an outer surface of the radially outermost positioned shell of the first 7 and second 8 shells, i.e. the second shell 8 for ease of reading, may comprise at least one peripherally extending, radially directed groove 13. As shown in FIG. 10c, the radial depth D of the groove 13 decreases in a direction towards the open end of the shell, e.g. open end 8a of second shell 8, such that the groove 13 comprises a maximum bottom thickness section T1 and a minimum bottom thickness section T2, the bottom thickness section being defined by the perpendicular distance between the outer surface and the inner surface of the shell 7, 8, 11, i.e. the thickness of the shell wall.

When the third shell 11 is in the unlocked position P1, the third shell 11 is at least partially arranged within the minimum bottom thickness section T2, as shown in FIGS. 6a and 6b. When the third shell 11 is in the locked position P2, the third shell 11 is at least partially arranged within the maximum bottom thickness section T1, as shown in FIGS. 7a and 7b, and the handle 2 is locked to the multiaxial joint 6.

In embodiments comprising the radially displaceable tongue 44, the tongue 44 is formed in the radially outermost positioned shell of the first 7 and second 8 shells. In the unlocked position P1 the radial position of the radially displaceable tongue 44 is unaffected by the third shell 11, and the first 7 and second 8 shells can be rotated in relation to each other. In the locked position P2 the radially displaceable tongue 44 has been moved towards a radially innermost positioned shell of the first 7 and second 8 shells, such that the first microstructure 43a and the second microstructure 43b engage in at least the common overlap area A. This engagement prevents the first 7 and second 8 shells from being rotated in relation to each other.

In one embodiment, the surgical instrument 1 further comprises a joint housing 14. The proximal end 14a of the joint housing 14 is connected to the handle 2 and the distal end 14b of the joint housing 14 is connected to the first 7 or the second 8 shell, preferably the outer second shell 8. The joint housing 14 is arranged such that it covers the multiaxial joint 6 and the proximal end 3a of the hollow shaft 3, see FIGS. 4a and 4b.

The joint housing 14 shares a center axis C with the hollow shaft 3, and the joint housing 14 is at least partially rotatable around the center axis C.

In one embodiment, shown in FIGS. 13a-13b and 14a-14c, the joint housing 14 comprises a flexible part 15 extending between at least first 16 and second 17 rigid parts, in the direction of the center axis C. The first rigid part 16 is connected to the first 7 or second 8 shell, and the second rigid part 17 is connected to the handle 2. As the hollow shaft 3 is rotated around the center axis C, the one of the rigid parts 16, 17 and a part of the flexible part 15 also rotate around the center axis C such that the joint housing 14 twists partially. The rigid parts are preferably made of a hard plastic such as e.g. ABS (acrylonitrile butadiene styrene) plastic. Thus, “rigid” as used herein is not an absolute rigidity and indicates a relatively high stiffness of the material, that is significantly higher than the stiffness of parts that are denoted as being “flexible”. The term “flexible” includes properties such as elasticity and bendability.

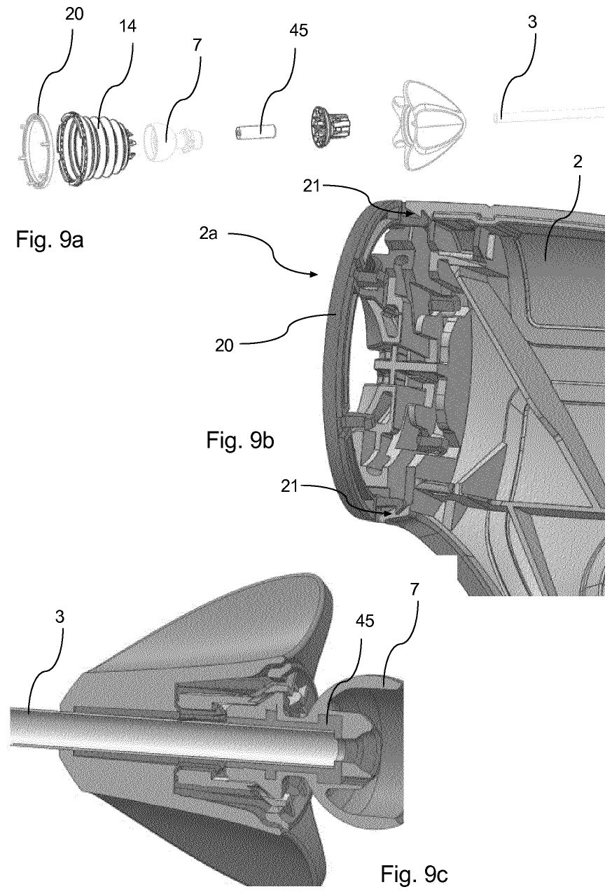

A proximal end 17a of the second rigid part 17 may comprise locking means 20 adapted for interacting with at least one corresponding recess 21 in the handle 2. As indicated in FIGS. 4b and 14a, locking means may be a peripheral flange 20 which interacts with recess 21. The locking means may also comprise a snap lock, as shown in FIGS. 9a and 9b.

At least one of the first 16 and second 17 rigid parts may comprise a plurality of grooves 18 extending in the direction of the center axis C, i.e. in a direction from the first rigid part 16 to the second rigid part 17. Each groove 18 comprises an end surface 18a extending at an angle to the center axis C, as shown in FIGS. 15a and 15b. The end surface 18a is arranged at a proximal end 16a of the first rigid part 16, while, correspondingly, a further end surface 18a is arranged at a distal end 17b of the second rigid part 17. The flexible part 15 fills the grooves 18 such that a flexible part surface 15a corresponding to the end surface 18a is formed in each groove 18.

Each end surface 18a comprises an opening 19 through which the flexible part 15 extends, the opening 19 extending generally parallel with the center axis C.

A section 15b of the flexible part may extend on a radially outer side and/or a radially inner side of the first rigid part 16, and a section 15c of the flexible part 15 may correspondingly extend on a radially inner side and/or a radially outer side of the second rigid part 17. In one embodiment, the section of the flexible part 15 extends on the radially outer side as well as the radially inner side of the first rigid part 16, such that the two sections of the flexible part 15 extend in parallel, separated by the wall of the first rigid part 16.

In one embodiment, at least one of the first 16 and second 17 rigid parts have a truncated cone shape, the first rigid part 16 having an outer diameter and/or an inner diameter which is smaller than the outer diameter and/or an inner diameter of the second rigid part 17.

The handle 2 and the operating means 5 are configured to actuate the end effector 4 between any possible open position OP and a closed position

CP, i.e. from an open position to the closed position as well as from the closed position to an open position.

The end effector 4 comprises a first leg 22 and a second leg 23, the second leg 23 being pivotally connected to the first leg 22 at the distal end of the end effector 4b. As shown in FIGS. 16a and 16b, the second leg 23 extends at an acute angle a to the first leg 22 when the end effector 4 is in the open position OP. As shown in FIGS. 16c, 16d, and 17b, the second leg 23 extends generally parallel with the first leg 22 when the end effector 4 is in the closed position CP. In other words, the first leg 22 is stationary in relation to the hollow shaft 3, while the second leg 23 is pivotable in relation to the first leg 22 and the hollow shaft 3.

As shown in FIGS. 17a and 18a, the distal end 23b of the second leg 23 is pivotally connected to the distal end 22b of the first leg 22 such that the second leg 23 is superimposed over the first leg 22 in the closed position CP. The pivotal connection is arranged at an end 4b of the end effector 4 which farthest away from the handle 2, and hence the individual operating the handle 2. When the end effector 4 is actuated from the open position OP to the closed position CP, the proximal end 23a of the second leg 23 is moved at least partially towards the handle 2.

In one embodiment, as shown in FIG. 17a, the second leg 23 comprises cutting means 24 for cutting tissue and the first leg 22 comprises a recess 25 for accommodating the cutting means when the end effector 4 is in the closed position CP.

In a further embodiment, the second leg 23 comprises a staple magazine.

As shown in FIGS. 17a and 18a, the distal end 4b of the end effector 4 may comprise protection lips 26 extending between the first leg 22 and the second leg 23, the protection lips 26 preventing tissue from getting stuck in the pivot connection connecting the first leg 22 and the second leg 23.

At least one support rod 27 may be connected to the end effector 4, as shown in FIGS. 18a and 18b. The proximal end 27a of the support rod 27 is pivotally connected to the proximal end 4a of the end effector 4, such that the support rod 27 extends at an acute angle 13 to the first leg 22 when the end effector 4 is in the open position OP, allowing the first leg 22, the second leg 23, and the support rod 27 to enclose a triangular space which may contain tissue. The support rod 27 extends in parallel with the first leg 22 when the end effector 4 is in the closed position CP.

In one embodiment, the operating means 5 comprises an axially extending rod 28, extending in parallel with the center axis C of the hollow shaft 3, and a second pivot link 29. The rod 28 is partially enclosed by the first leg 22 of the end effector 4, preferably being arranged in a radially extending recess in the first leg 22, as shown in FIGS. 20a-20c.

The distal end 28b of the rod 28 is coupled to the distal end 23b of the second leg 23 by means of the second pivot link 29, the second pivot link 29 extending at an angle to the axial extension of the rod and at an angle to the second leg 23. The proximal end 28a of the rod 28 is operably connected to the handle 2. The end effector 4 is actuated from the open position OP to the closed position CP when the rod 28 is moved in a direction away from the distal end 4b of the end effector 4. Correspondingly, the end effector 4 is actuated from the closed position CP to the open position OP when the rod 28 is moved in a direction towards the distal end 4b of the end effector 4.

The operating means 5 may further comprise a piston 30 and a spring 31, the rod 28 being partially enclosed by the piston 30 and the spring 31. Preferably, a proximal section of the rod 28 is enclosed by the first leg 22, and a distal section of the rod 28 is enclosed by the piston 30 and the spring 31. The spring 31 is arranged between the piston 30 and the handle 2 and/or the multiaxial joint 6. The spring 31 applies a force onto the proximal end 30a of the piston 30 in a direction towards the distal end 4b of the end effector 4.

The rod 28 and the piston 30 are axially moveable in relation to at least one of each other and the end effector 4. Furthermore, the rod 28 and the piston 30 are releasably connected such that the rod 28 and the piston 30 are axially moveable as one unit, in relation to the end effector 4. The rod 28 and the piston 30 may be releasably connected by means of a piston lock, as shown in FIGS. 20b, 21a and 21c, the piston lock comprising a plurality of locking grooves 32 arranged in pairs in the rod 28 and a corresponding locking protrusion 33 arranged at a distal end 30b of the piston 30.

The present disclosure further relates to a surgical system comprising the above discussed surgical instrument 1 and at least one ligation clip 34. A ligation clip, also known as a clamp, is e.g. used for ligating flow of bodily fluids within tissue, see FIG. 19.

The end effector 4 of the surgical instrument 1 is configured to enclose the ligation clip 34 such that the distal end 34b of the ligation clip 34 is arranged adjacent the distal end 4b of the end effector 4 and the proximal end 34a of the ligation clip 34 is arranged adjacent the proximal end 4a of the end effector 4, as shown in FIG. 21a.

The ligation clip 34 comprises a first ligation clip leg 35 and a second ligation clip leg 36, the first ligation clip leg 35 and the second ligation clip leg 36 being pivotally connected at the distal end 34b of the ligation clip 34. The first ligation clip leg 35 and the second ligation clip leg 36 comprise locking means 37 for interlocking the first 35 and second 36 ligation clip legs at the proximal end 34a of the ligation clip 34 in a position where the first ligation clip leg 35 and the second ligation clip leg 36 are superimposed onto each other.

When the end effector 4 is in an initial open position OP, and the ligation clip 34 is enclosed by the first leg 22 and the second leg 23 of the end effector 4, the ligation clip 34 has an open configuration where the second ligation clip leg 36 extends at an acute angle y to the first ligation clip leg 35. When the end effector 4 subsequently is actuated to a closed position CP, the configuration of the ligation clip 34 changes to a closed configuration where the first 35 and second 36 ligation clip legs are superimposed onto each other. As the end effector 4 is returned to the open position OP, the ligation clip 34 remains in the closed configuration by means of the locking means 37.

The surgical system may comprise a plurality of individual ligation clips 34, all in open configuration and arranged successively between the distal end 4b of the end effector 4 and the handle 2 along the rod 28 and/or the first leg 22 of the end effector 4. All ligation clips 34-X, except a first ligation clip 34-1 located adjacent the distal end 4b of the end effector 4 and enclosed by the first leg 22 and the second leg 23 of the end effector 4, are enclosed by the first leg 22 of the end effector 4 and the rod 28.

The ligation clips 34 are arranged in abutment with each other such that axial displacement of any one of the ligation clips 34-X, enclosed by the first leg 22 of the end effector 4 and the rod 28, induces a corresponding displacement of all ligation clips 34 located between the ligation clip 34-X and the distal end 4b of the end effector 4. In other words, all ligation clips 34 located closer to the distal end 4b of the end effector 4 than the ligation clip 34-X are pushed in a direction towards the distal end 4b by means of the ligation clip 34-X.

The present disclosure further relates to a method of operating the above described surgical system, shown in FIGS. 21b to 21h, wherein a first ligation clip 34-1 is arranged within the end effector 4 such that the first ligation clip 34-1 is enclosed by the first leg 22 of the end effector 4 and the rod 28, and the first ligation clip 34-1 has an open configuration.

The first ligation clip 34-1 is displaced in the direction towards the distal end 4b of the end effector 4, to a position where the first ligation clip 34-1 is enclosed by the first 22 and second 23 legs of the end effector 4 and the end effector 4 is in an open position OP, by moving the rod 28 in a direction towards the distal end 4b of the end effector 4, as shown in FIG. 21a.

The first ligation clip 34-1 is closed by actuating the end effector 4 to the closed position CP by moving the rod 28 in the direction away from the distal end 4b of the end effector 4.

Subsequently the rod 28 is moved in a direction towards the distal end 4b of the end effector 4, such that the end effector 4 is returned to the open position OP, which allows the first ligation clip 34-1, having a closed configuration, to be released from the end effector 4.

The surgical system may comprise a plurality of ligation clips 34. For ease of reading, the following description will be limited to a first, a second, and a third ligation clip 34, however additional ligation clips 34 are possible.

A second ligation clip 34-2 may be arranged within the end effector 4 such that the second ligation clip 34-2 is enclosed by the first leg 22 of the end effector 4 and the rod 28, the second ligation clip 34-2 having an open configuration. Furthermore, the piston lock 32, 33 interlocks the rod 28 and the piston 30 in the first locking position LP1 when the end effector 4 is returned to the open position OP, as mentioned above and shown in FIG. 21a.

The rod 28 is moved in the direction away from the distal end 4b of the end effector 4, as shown in FIG. 21b, whereby the piston lock 32, 33 is released from the first locking position LP1 and the piston 30 is allowed to move in relation to the rod 28. Simultaneously, the end effector 4 is actuated to the closed position CP, and the piston 30 moves towards the end effector 4, by means of the spring 31 applying force onto the proximal end 30a of the piston 30 until the piston lock 32, 33 engages the second locking position LP2, as shown in

FIG. 21c. The distal end 30b of the piston 30 is arranged closer to the distal end 4b of the end effector 4 when the piston lock 32, 33 is in the second locking position LP2 than in the first locking position LP1.

The rod 28 is moved in the direction towards the distal end 4b of the end effector 4, and the piston 30 is moved along with the rod 28 in the same direction. Simultaneously, the end effector 4 is actuated to the open position OP, as shown in FIG. 21d.

The rod 28 is moved in the direction away from the distal end 4b of the end effector 4, whereby the piston lock 32, 33 is released from the second locking position LP2, as shown in FIG. 16e. The locking protrusion 33 engages a longitudinal groove 38 arranged in the rod 28 such that the piston 30 is allowed to move in relation to the rod 28, simultaneously actuating the end effector 4 to the closed position CP, as shown in FIG. 21f.

The rod 28 and piston 30 may comprise only one piston lock 32, 33 and one longitudinal groove 38. However, the rod 28 preferably comprises several longitudinal grooves 38 as well as several individual or pairs of locking grooves 32, each pair allowing the locking protrusion 33 of the piston 30 to assume a first locking position LP1 when engaging one locking groove of the pair, and assume a second locking position LP2 when engaging the other locking groove of the pair. Each longitudinal groove 38 is associated with one individual or one pair of locking grooves 32.

The distal end 30b of the piston 30 engages with the second ligation clip 34-2, pushing the second ligation clip 34-2 in the direction towards the distal end 4b of the end effector 4 by means of the spring 31, until the second ligation clip 34-2, or a third ligation clip 34-3 located between the second ligation clip 34-2 and the distal end 4b of the end effector 4, is enclosed by the first 22 and second 23 legs of the end effector 4, as shown in FIG. 21g. The ligation clips 34 slide over the longitudinal grooves 38 such that the longitudinal grooves 38 and the ligation clips 34 do not interact.

The rod 28 is moved in the direction towards the distal end 4b of the end effector 4, simultaneously actuating the end effector 4 to the open position OP, as shown in FIG. 21h.

The second 34-2 or third 34-3 ligation clip, enclosed by the first 22 and second 23 legs of the end effector 4, is closed by actuating the end effector 4 to the closed position CP by moving the rod 28 in the direction away from the distal end 4b of the end effector 4.

The rod 28 is moved in the direction towards the distal end 4b of the end effector 4, such that the end effector 4 is returned to the open position OP, which allows the second 34-2 or third 34-3 ligation clip, being in the closed configuration, to be released from the end effector 4.

FIGS. 16a-d, 21a-d, and 22a-d show an embodiment of the surgical instrument 1 for actuating the end effector 4 between open and closed positions. The hollow shaft 3 is configured to guide the rod 28 for translative movement of the rod 28. the second leg 23 is pivotally suspended from the hollow shaft 3 at a first position on the second leg 23. The second leg 23 has a free proximal end 23a and a hinged distal end 23b, and the first position is at or adjacent the hinged distal end 23b.

The second pivot link 29 operatively connects the rod 28 with the second leg 23 by a first end 29a of the second pivot link 29 being pivotally connected to the axially extending rod 28 and a second end 29b of the second pivot link 29 being pivotally connected to the second leg 23 at a second position spaced from the first position. In one embodiment, the second pivot link 29 comprises a plate extending between the first end 29a and the second end 29b.

The first end 29a is connected to the rod 28 by a first hinge pin 40. The second pivot link 29 may be operatively connected to the rod 28 at or near an extremity of the rod 28, such as the proximal end 28a or the distal end 28b of the rod 28. Correspondingly, the second end 29b may be connected to the second leg 23 by a second hinge pin 41. The second position is nearer to the hinged distal end 23b then to the free proximal end 23a.

The second leg 23 is, in one embodiment, hinged to the hollow shaft 3 by a third hinge pin 42. The third hinge pin 42 is arranged in or adjacent the center axis C of the hollow shaft 3.

The second leg 23 is configured to rotate towards the hollow shaft 3 when the rod 28 is translated in a direction away from the third hinge pin 42 and the second leg 23 is configured to rotate away from the hollow shaft 3 when the rod 28 is translated in a direction towards the third hinge pin 42.

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

The reference signs used in the claims shall not be construed as limiting the scope.

1-47. (canceled)

48. A surgical instrument comprising
a handle,
a hollow shaft,
an end effector coupled to a distal end of said hollow shaft, said end effector being operatively connected to said handle through operating means extending within said hollow shaft, and
a multiaxial joint coupling a distal end of said handle to a proximal end of said hollow shaft, said multiaxial joint allowing multiaxial rotation of said hollow shaft in relation to said handle,
said multiaxial rotation including rotation of said hollow shaft around a center axis of said hollow shaft, and rotation in at least two planes in relation to said distal end of said handle.

49. The surgical instrument according to claim 48, wherein said hollow shaft is rotatable around said center axis and in three planes simultaneously.

50. The surgical instrument according to claim 48, wherein said operating means comprises an axially extending rod and a first pivot link, said first pivot link being arranged within an interior of said handle, a proximal end of said rod being connected to a proximal end of said first pivot link, said proximal end of said first pivot link further being slidingly connected to a grip part of said handle, and a distal end of said first pivot link being pivotally connected to an actuating part of said handle.

51. The surgical instrument according to claim 50, wherein said operating means further comprises a piston and a spring, said rod being partially enclosed by said piston and said spring, said spring applying a force onto a proximal end of said piston in a direction towards a distal end of said end effector, said rod and said piston being axially moveable in relation to at least one of each other and said end effector.

52. The surgical instrument according to claim 51, wherein said rod and said piston are releasably connected by means of a piston lock, said piston lock comprising a plurality of locking grooves arranged in said rod, and a corresponding locking protrusion arranged at a distal end of said piston.

53. The surgical instrument according to claim 48, wherein said multiaxial joint is a spherical joint comprising first and second at least partially spherical shells, open ends of said first and second shells having mating surfaces such that there is a common overlap area, comprising said open ends, between said first shell and said second shell when an open end of one of said first shell and said second shell is pressed into the open end of the other of said first shell and said second shell.

54. The surgical instrument according to claim 53, wherein at least one of said first and second shells is provided with at least one slit extending from said open end of said first and/or second shells.

55. The surgical instrument according to claim 53, wherein at least one mating surface of said first and second shells is provided with at least one of a microstructure, a coating, and a roughening for increased friction in said common overlap area,
wherein, optionally, said mating surface of said first shell is provided with a first microstructure and said mating surface of said second shell is provided with a second microstructure, said first microstructure and said second microstructure comprising one of a plurality of recesses and at least one protrusion, said protrusion(s) being adapted for engaging and interlocking with said recesses.

56. The surgical instrument according to claim 55, wherein one of said first microstructure and said second microstructure is provided on a radially displaceable tongue.

57. The surgical instrument according to claim 53, wherein said multiaxial joint further comprises a third shell arranged around a periphery of said first and second shells, in said common overlap area,
a locking lever protruding from said third shell,
said third shell being moveable between an unlocked position and a locked position by operating said locking lever.

58. The surgical instrument according to claim 57, wherein, in said unlocked position, diameters of said first and second shells are unaffected by said third shell allowing said first and second shells to be rotated in relation to each other,
and wherein, in said locked position, said diameters of at least one of said first and second shells are reduced in at least said common overlap area such that said first and second shells cannot be rotated in relation to each other.

59. The surgical instrument according to any one of claims 53, further comprising a joint housing, a proximal end of said joint housing being connected to said handle and a distal end of said joint housing being connected to said first or said second shell, said joint housing covering said multiaxial joint and said proximal end of said hollow shaft.

60. The surgical instrument according to claim 48, wherein said handle and said operating means are configured to actuate said end effector between an open position and a closed position,
a proximal end of said end effector being coupled to said distal end of said hollow shaft, said end effector comprising a first leg and a second leg, said second leg being pivotally connected to said first leg at said distal end of said end effector,
said second leg extending at an acute angle to said first leg when said end effector is in said open position, and said second leg extending generally parallel with said first leg when said end effector is in said closed position.

61. A surgical system comprising a surgical instrument according to claim 60 and at least one ligation clip, the end effector of said surgical instrument being configured to enclose said ligation clip such that a distal end of said ligation clip is arranged adjacent the distal end of said end effector and a proximal end of said ligation clip is arranged adjacent the proximal end of said end effector, said ligation clip comprising a first ligation clip leg and a second ligation clip leg, said first ligation clip leg and said second ligation clip leg being pivotally connected at said distal end of said ligation clip, said first ligation clip leg and said second ligation clip leg comprising locking means for interlocking said first and second ligation clip legs at said proximal end of said ligation clip in a position where said first ligation clip leg and said second ligation clip leg are superimposed onto each other, wherein, when said end effector is in an initial open position, said ligation clip has an open configuration where said second ligation clip leg extends at an acute angle to said first ligation clip leg, when said end effector has been actuated to the closed position, said ligation clip has a closed configuration where said first and second ligation clip legs are superimposed onto each other, when said end effector is returned to the open position, said ligation clip remains in said closed configuration by means of said locking means.

62. The surgical system according to claim 61, further comprising a plurality of ligation clips, in open configuration and arranged successively between said distal end of said end effector and the handle of said surgical instrument, all ligation clips but a first ligation clip located adjacent said distal end of said end effector being enclosed by the first leg of said end effector and the rod of said surgical instrument.

63. The surgical system according to claim 62, wherein said ligation clips are arranged in abutment such that axial displacement of any ligation clip enclosed by said first leg and said rod induces a corresponding displacement of all ligation clips located between said ligation clip and said distal end of said end effector.
